(19) United States
(12) Patent Application Publication
(10) Pub. No.: US 2026/0122134 A1
(43) Pub. Date: Apr. 30, 2026

(54) MRI System Monitoring with Cloud-Enabled Abnormality Prediction

(71) Applicant: Siemens Healthineers AG, Forchheim (DE)

(72) Inventors: Hai Ning Yang, Shenzhen (CN); Lei Xu, Shenzhen (CN)

(73) Assignee: Siemens Healthineers AG, Forchheim (DE)

(21) Appl. No.: 19/370,332

(22) Filed: Oct. 27, 2025

(30) Foreign Application Priority Data
Oct. 28, 2024 (CN) 202411518645.4

(51) Int. Cl. H04L 67/12; G16H 40/40; G16H 40/67

(52) U.S. Cl. CPC H04L 67/12; G16H 40/40; G16H 40/67

(57) ABSTRACT

A monitoring method and system for a magnetic resonance imaging (MRI) system, and a magnetic resonance imaging system. The method includes: obtaining monitoring data of at least one monitoring target in an MRI system; converting the monitoring data of the at least one monitoring target into wireless communication protocol data, and transmitting the wireless communication protocol data to a cloud server, so that, for each monitoring target of the at least one monitoring target, the cloud server tracks a trend of change of monitoring data of the target and performs a prejudgment on an abnormality that may occur, and, when judging that an abnormality may occur, transmits warning information to a user.

### TECHNICAL FIELD

The present disclosure relates to the technical field of magnetic resonance imaging (MRI), specifically to a monitoring method and system for a magnetic resonance imaging system, and a magnetic resonance imaging system.

### BACKGROUND

MRI systems are based on magnetic fields generated by magnets and use the phenomenon of nuclear magnetic resonance to obtain information on molecular structure and the internal structure of the human body, etc.

At present, monitoring data of an MRI system are all transmitted to a dedicated aftersales service server by means of a hospital network and the monitoring thereof is the responsibility of a specialist; if the MRI system encounters a problem, such as a cooling water abnormality, a compressor stopping or a superconducting magnet overheating, then a worker at the dedicated aftersales service server end manually calls a user by means of a service hotline to resolve the problem. However, after work or during vacation time, if the MRI system encounters a problem, it is generally not possible to notify a user in time to adopt preventative measures to avoid a worse outcome, and therefore, the user has no choice but to deal with the problem that has already occurred and any serious damage caused thereby. In addition, since dedicated aftersales service servers are normally deployed in an external network, when monitoring data of the MRI system are transmitted by means of the hospital network, there is a risk that patient information that is stored in the hospital network is leaked to the external network, and therefore patient security cannot be guaranteed.

For this reason, those skilled in the art are still endeavoring to find other solutions for magnetic resonance imaging systems.

### SUMMARY

In view of this, aspects of the present application propose a monitoring method for a magnetic resonance imaging system, a monitoring system for a magnetic resonance imaging system, and a magnetic resonance imaging system, used to promptly ascertain a potential problem with an MRI system, avoiding unnecessary damage.

Aspects of the present application propose a monitoring method for a magnetic resonance imaging system, comprising: obtaining monitoring data of at least one monitoring target in an MRI system; converting the monitoring data of the at least one monitoring target into wireless communication protocol data, and sending the wireless communication protocol data to a cloud server, so that, for each monitoring target of the at least one monitoring target, the cloud server tracks a trend of change of monitoring data of the target and performs a prejudgment on an abnormality that may occur, and, when judging that an abnormality may occur, sends corresponding warning information to a user.

In one aspect, the at least one monitoring target comprises at least one or any combination of the following targets: a cooling system, a compressor, a magnet, a coil, and a system power source.

In one aspect, the step of obtaining monitoring data of at least one monitoring target in the MRI system comprises: a data transmission module obtaining monitoring data of at least one monitoring target in the MRI system by means of a system bus.

In one aspect, the data transmission module is a 4G data transmission unit, DTU, or a WIFI module.

In one aspect, the data transmission module is arranged in an equipment room that is isolated from a scanning room of the MRI system.

In one aspect, the step of, for each monitoring target of the at least one monitoring target, tracking a trend of change of monitoring data of the target and performing a prejudgment on an abnormality that may occur, comprises: at least one or any combination of the following: judging whether a temperature of cooling water of a cooling system exceeds a first warning threshold value; judging whether a flow rate of a compressor is reduced to a second warning threshold value; judging whether a magnet is at risk of losing superconductivity; judging whether a temperature of a coil is raised above a third warning threshold value; judging whether a system power source experiences undervoltage or overvoltage on the basis of a preset undervoltage threshold value and overvoltage threshold value.

In one aspect, the step of sending corresponding warning information to a user comprises: sending warning information that indicates a corresponding abnormality to a user by means of a short message, telephone, WeChat, or another application program or mini program.

In one aspect, this method further comprises: the cloud server receiving an inspection request from a user, and, according to the inspection request, presenting received monitoring data of each monitoring target in the MRI system to the user.

Aspects of the present application propose a monitoring system for a magnetic resonance imaging system, comprising: a data transmission module, which is used for obtaining monitoring data of at least one monitoring target in an MRI system, converting the monitoring data of the at least one monitoring target into wireless communication protocol data, and sending the wireless communication protocol data to a cloud server; and a cloud server, which, for each monitoring target of the at least one monitoring target, is used for tracking a trend of change of monitoring data of the target and performing a prejudgment on an abnormality that may occur, and, when judging that an abnormality may occur, sending corresponding warning information to a user.

In one aspect, the data transmission module is arranged in an equipment space that is isolated from a scanning space of the MRI system.

In one aspect, the cloud server comprises: a data receiving module, a data processing module, a warning module and a user interface module, wherein the data receiving module is used for receiving monitoring data of at least one monitoring target in an MRI system from the data transmission module, and providing the monitoring data to the data processing module after parsing; the data processing module, for each monitoring target of the at least one monitoring target, is used for tracking a trend of change of monitoring data of the target and performing a prejudgment on an abnormal problem that may occur, and, when judging that an abnormality may occur, providing warning information that indicates the corresponding abnormality to the warning module; the warning module is used for sending the warning information to a user according to a preset communication mode; the user interface module is used for receiving inspection information from a user, and, according to the inspection information, presenting monitoring data of each monitoring target in the MRI system from the data receiving module to the user.

Aspects of the present application propose a magnetic resonance imaging system, comprising: the monitoring system for a magnetic resonance imaging system described above.

From the above solutions, it can be seen that, in the technical solutions in the aspects of the present application, monitoring data of at least one monitoring target in an MRI system are sent to a cloud server, and the cloud server tracks a trend of change of the monitoring data, and, on the basis of the trend of change, performs a prejudgment on an abnormality that the MRI system may produce, and, when judging that an abnormality may occur, automatically sending warning information to a user, such that the user may promptly know of the possible abnormality in advance and perform corresponding processing.

In addition, by means of arranging a data transmission module, which converts and sends wireless data, in an equipment space that is remote from an MRI system scanning space, this wireless data transmission does not affect the imaging quality of the MRI system.

### BRIEF DESCRIPTION OF THE DRAWINGS

Preferred aspects of the present application are described in detail below with reference to the drawings, to give those skilled in the art a clearer understanding of the above and other features and advantages of the present application. In the drawings:

FIG. 1 is a demonstrative flowchart of a monitoring method for a magnetic resonance imaging system in an aspect of the present application.

FIG. 2 is a demonstrative structural drawing of a monitoring system for a magnetic resonance imaging system in an aspect of the present application.

### KEY TO THE DRAWINGS

| Label | Meaning |
|---|---|
| S11-S13 | steps |
| 21 | data transmission module |
| 22 | cloud server |
| 221 | data receiving module |
| 222 | data processing module |
| 223 | warning module |
| 224 | user interface module |
| 231, 232, 233, 234 | monitoring the target in the MRI system |

### DETAILED DESCRIPTION

In aspects of the present application, in order to be able to promptly ascertain the condition of an MRI system, the transmission of monitoring data of multiple targets in an MRI system to the cloud is considered, so that remote monitoring of the MRI system can be realized. For example, in an example, a data transmission unit (DTU), such as a 4G DTU, may convert monitoring data of an MRI system from a CAN bus format to a message queuing telemetry transport format (MQTT), and then sends the data to a cloud server. Next, the cloud server may monitor key parameters of the MRI system, track a trend of change of these parameters, and, according to the trend of change, perform a prediction on a problem that may occur, and when judging that an abnormal problem may occur, send warning information to a user.

To enable a clearer understanding of the object, technical solutions, and effects of the present application, particular aspects of the present application are now explained with reference to the drawings, in which identical labels indicate structurally identical components or components with similar structures but identical functions.

As used herein, “exemplary” and “schematic” mean “serving as an instance, example, or illustration”. No drawing or aspect described herein as “exemplary” or “schematic” should be interpreted as a more preferred or more advantageous technical solution.

To make the drawings appear uncluttered, only those parts relevant to the present application are shown schematically in the drawings; they do not represent the actual structure thereof as a product.

In this text, “a” does not only mean “just this one”; it may also mean “more than one”. In this text, “first”, “second”, etc., are merely used to differentiate between parts, not to indicate the order or degree of importance between parts, etc.

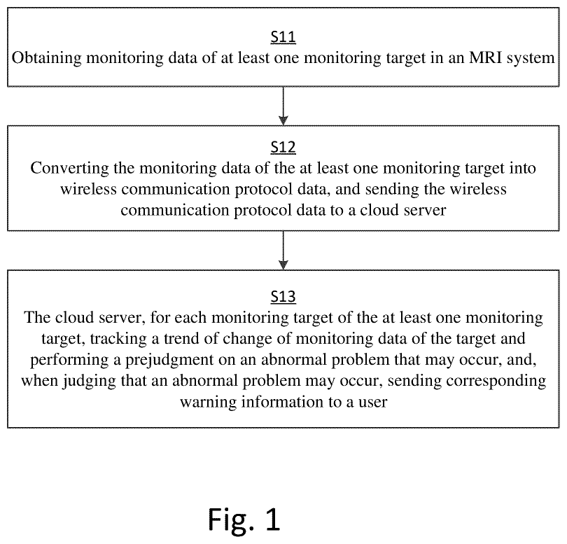

FIG. 1 is a demonstrative flowchart of a monitoring method for a magnetic resonance imaging system in an aspect of the present application. As FIG. 1 shows, the method may comprise the following steps:

step S11, obtaining monitoring data of at least one monitoring target in an MRI system.

In the present aspect, monitoring data of at least one monitoring target in an MRI system may comprise: monitoring data of a cooling system, monitoring data of a compressor, monitoring data of a magnet, monitoring data of a coil, monitoring data of a system power source, etc.

In an example, monitoring data of at least one monitoring target in an MRI system may be obtained by means of a system bus, such as a control area network (CAN) bus; for example, monitoring data of at least one monitoring target in an MRI system are obtained by a data transmission module, such as a 4G DTU or a WIFI module, etc., by means of a system bus, and this 4G DTU module or WIFI module may be arranged in an equipment space of an MRI system machine room; this equipment space is isolated from a scanning space in which the magnet system is located, and therefore sending a wireless data signal does not interfere with a magnetic field of the MRI system, so as not to affect imaging quality.

Step S12, converting the monitoring data of the at least one monitoring target into wireless communication protocol data, and sending the wireless communication protocol data to a cloud server.

In an example, a data transmission module, such as a 4G DTU module or a WIFI module, may convert data of a system bus format into wireless communication protocol data, such as data of an MQTT format, etc. The data of the system bus format may be data of a CAN bus format or data of another serial interface format, etc.

Step S13, the cloud server, for each monitoring target of the at least one monitoring target, tracking a trend of change of monitoring data of the target and performing a prejudgment on an abnormal problem that may occur, and, when judging that an abnormal problem may occur, sending corresponding warning information to a user.

For example, in an example, whether a temperature of cooling water of a cooling system exceeds a first warning threshold value may be judged; and/or whether a flow rate of a compressor is reduced to a second warning threshold value may be judged; and/or whether a magnet is at risk of losing superconductivity may be judged; and/or whether a temperature of a coil is raised above a third warning threshold value may be judged; and/or whether a system power source experiences undervoltage or overvoltage may be judged on the basis of a preset undervoltage threshold value and overvoltage threshold value, etc.

In an example, when it is judged that an abnormal problem may occur, warning information that indicates a corresponding abnormality may be sent to a user using a short message, telephone, WeChat, or another application program (app) or mini program, etc.

In addition, a user may use a mobile phone, a laptop computer, etc., to log in to a cloud server, to inspect monitoring data of each target in an MRI system at any time and from any place, thereby monitoring parameter status of each target. When specifically implemented, a user may log in to a cloud server by means of a corresponding application program (app) or another mini program or webpage, etc. That is, the cloud server may present received monitoring data of each monitoring target in the MRI system to the user according to the inspection information of the user.

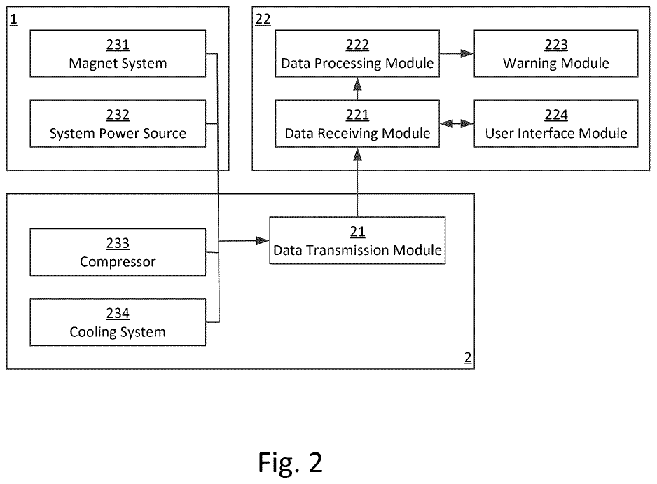

In addition, aspects of the present application further provide a monitoring system for a magnetic resonance imaging system, which is used for implementing the monitoring method for a magnetic resonance imaging system as shown in FIG. 1. For details that are not disclosed in the present system aspect, reference may be made to the corresponding description in the method aspect. FIG. 2 shows a demonstrative structural drawing of a monitoring system for a magnetic resonance system in an aspect of the present application. As shown in FIG. 2, this monitoring system may comprise: a data transmission module 21 and a cloud server 22.

The data transmission module 21 is used for obtaining monitoring data of at least one monitoring target in an MRI system, converting the monitoring data of the at least one monitoring target into wireless communication protocol data, and sending the wireless communication protocol data to a cloud server.

In the present aspect, the at least one monitoring target comprises: a magnet system 231 and a system power source 232 that are arranged in a scanning space 1, and a compressor 233 and a cooling system 234 that are arranged in an equipment space 2. In another aspect, at least one monitoring target may also comprise a different target to that shown in FIG. 2.

In an example, this data transmission module 21 may be a 4G DTU module, and this 4G DTU module may be arranged in the equipment space 2 of the MRI system; since the equipment space 2 is isolated from the scanning space 1, sending a wireless data signal does not affect the imaging quality of the MRI system.

The cloud server 22, for each monitoring target of the at least one monitoring target, is used for tracking a trend of change of monitoring data of the target and performing a prejudgment on an abnormality that may occur, and, when judging that an abnormality may occur, sending corresponding warning information to a user. In addition, the cloud server 22 may be further used for presenting received monitoring data of each monitoring target in the MRI system to the user according to the inspection information of the user.

In one aspect, the cloud server 22 comprises: a data receiving module 221, a data processing module 222, and a warning module 223. In addition, the cloud server may further comprise: a user interface module 224.

The data receiving module 221 is used for receiving monitoring data of at least one monitoring target in an MRI system from the data transmission module 21, and providing the monitoring data to the data processing module 222 after parsing.

The data processing module 222, for each monitoring target of the at least one monitoring target, is used for tracking a trend of change of monitoring data of the target and performing a prejudgment on an abnormality that may occur, and, when judging that an abnormality may occur, providing warning information that indicates the corresponding abnormality to the warning module 223.

The warning module 223 is used for sending the warning information to a user according to a preset communication mode.

The user interface module 224 is used for receiving inspection information from a user, and, according to the inspection information, presenting monitoring data of each monitoring target in the MRI system from the data receiving module 221 to the user. In addition, the communication mode and related threshold value information, etc., may also be preset by a user by means of this user interface module 224.

Aspects of the present disclosure further provide a magnetic resonance imaging system, which may comprise the monitoring system described above.

In aspects of the present application, monitoring data of at least one monitoring target in an MRI system are sent to a cloud server, and the cloud server tracks a trend of change of the monitoring data, and, on the basis of the trend of change, performs a prejudgment on an abnormality that the MRI system may produce, and, when judging that an abnormality may occur, automatically sends warning information to a user, such that the user may promptly know of the possible abnormality in advance and perform corresponding processing. The MRI system encountering a serious problem is avoided, reducing system downtime at the user site.

In addition, by means of arranging a data transmission module, which converts and sends wireless data, in an equipment space that is remote from an MRI system scanning space, this wireless data transmission does not affect the imaging quality of the MRI system.

The foregoing descriptions are only preferred aspects of the present application and are not intended to limit the present application. Any modifications, equivalent replacements, and improvements made without departing from the spirit and principle of the present application shall fall within the scope of protection of the present application.

1. A monitoring method for a magnetic resonance imaging (MRI) system, comprising:
obtaining monitoring data of at least one monitoring target in an MRI system; and
converting the monitoring data of the at least one monitoring target into wireless communication protocol data, and transmitting the wireless communication protocol data to a cloud server,
wherein for each monitoring target of the at least one monitoring target, the cloud server tracks a trend of changes in the monitoring data of the respective monitoring target, performs a prejudgment of a potential abnormality that may occur, and, when determining that an abnormality may occur, transmits corresponding warning information to a user.

2. The monitoring method of claim 1, wherein the at least one monitoring target comprises at least one or any combination of a cooling system, a compressor, a magnet, a coil, and a system power source.

3. The monitoring method of claim 2, wherein the step of obtaining monitoring data of at least one monitoring target in the MRI system comprises a data transmission module obtaining monitoring data of at least one monitoring target in the MRI system using a system bus.

4. The monitoring method of claim 3, wherein the data transmission module is a 4G data transmission unit (DTU) module or a WIFI module.

5. The monitoring method of claim 3, wherein the data transmission module is arranged in an equipment room that is isolated from a scanning room of the MRI system.

6. The monitoring method of claim 5, wherein the step of, for each monitoring target of the at least one monitoring target, tracking a trend of change of monitoring data of the target and performing a prejudgment on an abnormality that may occur, comprises: at least one or any combination of the following:
judging whether a temperature of cooling water of a cooling system exceeds a first warning threshold value;
judging whether a flow rate of a compressor is reduced to a second warning threshold value;
judging whether a magnet is at risk of losing superconductivity;
judging whether a temperature of a coil is raised above a third warning threshold value; and
judging whether a system power source experiences undervoltage or overvoltage based on a preset undervoltage threshold value and overvoltage threshold value.

7. The monitoring method of claim 1, wherein the step of transmitting corresponding warning information to a user comprises:
transmitting warning information that indicates a corresponding abnormality to a user using a short message, telephone, WeChat, or another application program or mini program.

8. The monitoring method of claim 1, wherein the method further comprises: the cloud server receiving an inspection request from a user, and, according to the inspection request, presenting received monitoring data of each monitoring target in the MRI system to the user.

9. A monitoring system for a magnetic resonance imaging system (MRI), comprising:
a data transmission module configured to obtain monitoring data of at least one monitoring target in an MRI system, convert the monitoring data of the at least one monitoring target into wireless communication protocol data, and transmit the wireless communication protocol data to a cloud server; and
a cloud server configured to, for each monitoring target of the at least one monitoring target, track a trend of change of monitoring data of the target and perform a prejudgment on an abnormality that may occur, and, when judging that an abnormality may occur, transmit corresponding warning information to a user.

10. The monitoring system of claim 9, wherein the data transmission module is arranged in an equipment room that is isolated from a scanning room of the MRI system.

11. The monitoring system of claim 9, wherein the cloud server comprises: a data receiving module, a data processing module, a warning module, and a user interface module, wherein:
the data receiving module is configured to receive monitoring data of at least one monitoring target in an MRI system from the data transmission module, and provide the monitoring data to the data processing module after parsing,
the data processing module, for each monitoring target of the at least one monitoring target, is configured to track a trend of change of monitoring data of the target and perform a prejudgment on an abnormality that may occur, and, when judging that an abnormality may occur, provide warning information that indicates the corresponding abnormality to the warning module,
the warning module is configured to transmit the warning information to a user according to a preset communication mode, and
the user interface module is configured to receive inspection information from a user, and, according to the inspection information, present monitoring data of each monitoring target in the MRI system from the data receiving module to the user.

12. A magnetic resonance imaging system, comprising: the monitoring system for a magnetic resonance imaging system of claim 9.
